(19) United States
(12) Patent Application Publication
(10) Pub. No.: US 2012/0323107 A1
(43) Pub. Date: Dec. 20, 2012

(54) Methods for Assessing Tophus Response During Urate Lowering Therapy in Treatment Tophaceous Gout

(75) Inventors: Allan Maroli, Beade Mead, NJ (US); Michelle Alton, Yardley, PA (US); Royce W. Waltrip, Cranbury, NJ (US); Claudia Rehrig, Plainsboro, NJ (US); Bill Huang, Florham Park, NJ (US); Zebulun Horowitz, Basking Road, NJ (US); Robert Ford, Belle Mead, NJ (US)

(73) Assignees: Corelab Partners, Inc., Princeton, NJ (US); Savient Pharmaceuticals, Inc., East Brunswick, NJ (US)

(21) Appl. No.: 13/379,719

(22) PCT Filed: Jun. 25, 2010
(86) PCT No.: PCT/US10/40093

§ 371 Date: Sep. 5, 2012

Related U.S. Application Data
(60) Provisional application No. 61/269,669, filed on Jun. 25, 2009.
(60) Provisional application No. 61/248,680, filed on Oct. 5, 2009.
(60) Provisional application No. 61/261,589, filed on Nov. 16, 2009.

(51) Int. Cl. A61B 6/00

(52) U.S. Cl. 600/407

(57) ABSTRACT

Systems and methods for assessment of the reduction of tophus burden with effective urate-lowering therapy were developed. The first application of computer-assisted methods for accessing tophus response included standardized digital photography, computer assisted measurement and analysis of tophi by an independent reader and blinded central reader paradigm. The ability of the method for computer-assisted analysis of digital photographs to quantify reduction in tophus size demonstrates its feasibility as a urate-lowering therapy monitoring tool in the treatment of tophaceous gout.

### CROSS REFERENCE TO RELATED APPLICATIONS

This application claims priority to and benefit of U.S. Provisional Application No. 61/269,669, filed on Jun. 25, 2009, U.S. Provisional Application No. 61/248,680, filed on Oct. 5, 2009, U.S. Provisional Application No. 61/261,589, filed on Nov. 16, 2009.

### FIELD OF THE INVENTION

This invention relates to methods accessing tophus elimination during urate lowering therapy in tophaceous gout.

### BACKGROUND OF THE INVENTION

Throughout this application, various publications are referenced within the text. The disclosure of these publications in their entireties are hereby incorporated by reference into this application in order to more fully describe the state of the art as known to those skilled in therein as of the date of the invention described and claimed herein.

Gout is a disease characterized in part by deposition of urate crystals in and around joints, producing in some patients tophaceous mass lesions that are visible, palpable, and measurable. Gout is the most prevalent form of arthritis in men and is increasing in incidence and prevalence among older persons of both genders. Chronic gout in which hyperuricemia is poorly managed with urate levels remaining above subsaturating levels, can result in severe outcomes of progressive disease characterized clinically by frequent acute and exquisitely painful gout flares lasting up to about 10 days, chronic painful gouty arthritis and chronic arthropathy; destructive tophi; impaired quality of life; and chronic disability.

The advanced nature of tophaceous gout requires modification in the pursuit of the historical goals of medical management of gout in order to reverse urate crystal deposition, eventually abolishing gouty signs and symptoms including tophaceous masses. The resolution of tophi is an important aim and can be accelerated by agents such as uricase therapies that have a mode of action that rapidly converts urate to the more soluble metabolite allantoin. Allantoin is more readily excreted from the body in urine than urate, and by this proposed mechanism, there is a reduction in the total body urate burden.

Elevated serum uric acid levels are a clear risk factor for gout. Humans, unlike most mammals, are susceptible to gout because they do not express the uricase enzyme that converts uric acid to a more soluble metabolite allantoin. Persistently elevated plasma uric acid (PUA) or serum uric acid (SUA) levels result in deposition of uric acid in joints and soft tissues. As the total body burden of uric acid increases, signs and symptoms of gout result, including development of tophi with resultant chronic pain/inflammation and consequent loss of physical function.

Effective urate-lowering therapies work by decreasing the level of urate circulating in the blood, thereby correcting the hallmark hyperuricemia in patients with gout. Urate lowering therapies include agents that block the uric acid metabolic pathway, such as allopurinol, an inhibitor of xanthine oxidase. Other urate-lowering therapies known as uricosurics work by increasing the rate of excretion of uric acid in the urine, such as probenecid.

Pegylated uricase, a recombinant, mammalian uricase modified by the covalent attachment of monomethoxy-poly(ethylene glycol)(PEG), is unique among urate-lowering therapies, converting urate, which is sparingly soluble in extracellular fluids to allantoin, a more soluble endproduct that is readily excreted by the kidney. Pegylated uricase treatment is designed to reduce and maintain blood concentrations of uric acid below limits of solubility (SUA or PUA <6 mg/dL). It is expected that, with the use of pegylated uricase over time, soluble and insoluble (crystalline) extracellular urate pools will decrease as a result of the induced concentration gradient between tissue stores and intravascular urate.

Based on the pharmacodynamic effect of urate lowering therapy on serum uric acid, it was expected that clinical benefit in the tophaceous gout population would be observed by complete resolution or decreases in number of tophi and improvements in patient reported global assessments of disease activity, pain, physical function and health-related quality of life. The tophus response was selected as one of the most important secondary outcome measures to demonstrate that sustained lowering of uric acid levels would be associated with clinically meaningful improvements. Thus, there is a need in the art for quantitative methods to assess tophus elimination and correlate the tophus response to pegylated uricase therapy.

### SUMMARY OF THE INVENTION

The present invention provides a method for accessing and/or quantitating tophus response during a urate lowering therapy in a patient comprising a) obtaining a photograph of the measurable baseline tophus before treatment, b) obtaining a photograph of the measurable baseline tophus after treatment, c) measuring the longest dimension and the perpendicular dimension of said tophus, d) calculating the area of said tophus by multiplying said longest dimension and said perpendicular dimension, e) comparing the tophus area before and after treatment; and indicating that a change from baseline in the area of the tophus after treatment correlates with therapeutic efficacy.

The present invention also provides for administration of pegylated uricase therapy to the patient. In one embodiment, pegylated uricase is administered to a patient at a dosage of 8 mg every 2 weeks. In another embodiment, the pegylated uricase is administered to a patient at a dosage of 8 mg every 4 weeks. In yet another embodiment, the pegylated uricase is administered to a patient at a dosage of 8 mg every 3 weeks.

The methods of the present invention include the tophus photographs taken after 25 weeks of pegylated uricase treatment. In one embodiment, the tophus paragraphs are taken after 13 weeks of the pegylated uricase treatment. In yet another embodiment, the tophus paragraphs are taken 19 weeks after pegylated uricase treatment.

The methods of the present invention relate to the patient who is suffering from tophaceous gout.

The methods of the present invention result in 100% decrease in the area of the tophus from baseline as indicative of complete response of the patient to pegylated uricase therapy. In one embodiment, at least 75% decrease in the tophus area from baseline correlates with marked response to pegylated uricase therapy. In another embodiment, at least 50% decrease in the tophus area from baseline correlates with partial response to pegylated uricase therapy. Finally, at least 25% increase in the tophus area from baseline correlates with progressive disease.

### BRIEF DESCRIPTION OF THE DRAWINGS

FIG. 1 depicts light stand with camera and template used to take photographs of hands and feet.

FIG. 2 depicts calibration rulers used for anatomic sites other than hands and feet.

FIG. 3A depicts the use of electronic calipers measuring the two longest diameters of measured tophi of a hand (perpendicular lines).

FIG. 3B depicts the use of electronic calipers measuring the two longest diameters of measured tophi of an elbow (perpendicular lines).

FIG. 4A shows serial photographs in patients at baseline.

FIG. 4B shows serial photographs in patients at week 13 of pegylated uricase treatment depicting tophus resolution.

FIG. 4C shows serial photographs in patients at week 19 of pegylated uricase treatment depicting tophus resolution.

FIG. 4D shows serial photographs in patients at week 25 of pegylated uricase treatment depicting tophus resolution.

### DETAILED DESCRIPTION OF THE INVENTION

It had been surprisingly discovered that three-dimensional tophaceous lesions can be systematically and quantitatively measured using non-invasive two-dimensional imaging techniques.

### DEFINITIONS

In accordance with this detailed description, the following abbreviations and definitions apply. It must be noted that as used herein, the singular forms “a,” “an,” and “the” include plural referents unless the context clearly dictates otherwise.

The term “therapeutic efficacy” as used herein refers to the effectiveness of a particular treatment regimen. Specifically, therapeutic efficacy is defined by achieving serum urate levels less than or about 6 mg/dl. This includes a balance of efficacy, toxicity (e.g., side effects and patient tolerance of a formulation or dosage unit), patient compliance, and the like.

The terms “treating,” “treatment,” and the like are used herein to refer to obtaining a desired pharmacological and physiological effect. The effect may be prophylactic in terms of preventing or partially preventing a disease, symptom, or condition thereof and/or may be therapeutic in terms of a partial or complete cure of a disease, condition, symptom, or adverse effect attributed to the disease. The term “treatment,” as used herein, covers any treatment of a disease in a mammal, such as a human, and includes: (a) preventing the disease from occurring in a patient which may be predisposed to the disease but has not yet been diagnosed as having it, i.e., causing the clinical symptoms of the disease not to develop in a patient that may be predisposed to the disease but does not yet experience or display symptoms of the disease; (b) inhibiting the disease, i.e., arresting or reducing the development of the disease or its clinical symptoms; and (c) relieving the disease, i.e., causing regression of the disease and/or its symptoms or conditions.

Treating a patient's suffering from disease related to pathological inflammation is contemplated. Preventing, inhibiting, or relieving adverse effects attributed to pathological inflammation over long periods of time and/or are such caused by the physiological responses to inappropriate inflammation present in a biological system over long periods of time are also contemplated.

The term CAPER refers to (Computer-Assisted Photographic Evaluation in Rheumatology), which was created to provide categorical scoring of tophus response recorded by photographic imaging.

Current urate-lowering management involves nonpharmacological and pharmacological strategies. The non-pharmacological strategies aim for urate lowering through lifestyle alterations, such as changes in diet composition and quantity, weight loss, reduction in alcohol consumption, and dietary supplementation such as with vitamin C. Lifestyle initiatives for gout patients are beneficial not only by reducing SUA but also by addressing risk factors contributing to comorbidities in gout patients.

Even when lifestyle alterations can be adhered to, however, they often do not provide sufficient urate lowering to control established gout, and pharmacological management is ultimately indicated.

Among the pharmacological urate-lowering strategies are treatments with uricosuric agents to promote renal uric acid excretion or, much more commonly, the purine analogue xanthine oxidase inhibitor, allopurinol, to reduce urate production. Among the new pharmacological agents are nonpurine analogue xanthine oxidase inhibitor, febuxostat, and pegylated uricase.

Tophus photography is a key procedure in evaluating efficacy of pegylated uricase in this trial. Therefore, special care has been taken not only in the execution of the photographs but also in the processing of the Digital Media Storage Card containing the photographic data. The following assembly and preparation steps are preferably undertaken before a photograph had been taken: 1) place the base on a flat table; 2) push the stanchion through the light fixture bracket and light assembly bracket on the bottom; 3) center the stanchion and light bracket over the square receptacle on the base; 4) affix the light assembly mounting arms to the light assembly bracket; 5) slide the camera holder over the upright stanchion and slide it down so that the holder is at the 18″mark; 6) attach the camera to the camera holder by aligning the threads on the holder with the threaded hole on the bottom of the camera and 7) screw the light bulbs in and test the lights to ensure they work. Turn the lights off and slide the light fixture so they are in line with the camera.

Patients can have one or multiple tophus lesions. Tophi are categorized as measurable or unmeasured, where unmeasured refers to a semi-quantitative assessment of change rather than accurate measurement of linear dimensions. To be considered measurable, tophi are preferably ≧5 mm in the longest dimension at baseline, and must have borders distinguishable to the trained central reader. At baseline up to 5 measurable tophi, preferentially from the hands and feet, in the photographs are chosen and measured bidimensionally by a central reader. Any measurable tophi beyond the 5 chosen can be considered unmeasured. Up to 2 tophi that are representative of the patient's tophus burden but which cannot be accurately measured (e.g. due to location, shape or other factors) were followed during the study.

The unmeasured tophi are preferably approximately 10 mm or greater at baseline in order for the reader to reliably assess qualitative changes. Up to 2 tophi that are representative of the patient's tophus burden but which either cannot be accurately measured (e.g. due to location, shape or other factors) or are in excess of the initial 5 selected as “measurable” tophi, can be followed.

Selected tophi are preferably representative of the patient's overall tophus burden. Baseline photographs are used to prospectively identify sites of tophaceous disease. All other sites not chosen as measurable tophi are characterized as unmeasured tophi as defined above. Measurable tophi are preferentially chosen from the hands and feet because photographs of these areas are more readily standardized. For any measurable tophus at any time point, measurements are taken and recorded bi-dimensionally. The longest dimension (LD) is preferably recorded first. The perpendicular measurement is preferably recorded second. The bi-dimensional measurements of each measurable tophus are multiplied to obtain the area of each measurable tophus. The baseline area of each measurable tophus is used as reference to characterize the objective response of each measurable tophus. Measurable tophi are measured at each time point. Where there are no measurable tophi identified, photographic assessments are based on unmeasured tophi.

Measurements can be performed using electronic calipers on digital images, viewed on computer screen. Measurements (LD, width, and the area) are captured in a database. Tophi can be measured and reported in millimeters.

Further, the response assessment for each individual measurable tophi is compared to the area of the measurable tophus at baseline and defined as follows: 100% decrease in the area of the tophus is a complete response, at least 75% decrease in the area of the tophus is a marked response, at least 50% decrease in the area of the tophus is partial response, and neither 50% decrease nor a 25% decrease in the area of the measurable tophus is referred to as stable disease. Progressive disease is referred to as a 25% or more increase in the area of the tophus. Tophi that cannot be evaluated are captured as UE (unevaluable).

For the unmeasured tophi, the evaluation is conducted where up to two unmeasured tophi are semi-quantitatively evaluated at each time point. Response assessment for each individual unmeasured tophus is compared to semi-quantitative assessment of the unmeasured tophus at baseline and defined as follows: Complete response (CR)—complete disappearance of the tophus; Improved (I)—an approximate 50% or more reduction in the size of the tophus; Stable disease (SD)—neither improvement (I) nor progression (PD) can be determined; Progressive disease (PD)—an approximately 50% or more increase in the size of the tophus; and Unable to evaluate (UE)—unable to assess for any reason at a given time point.

The urate lowering therapy of the present invention is useful for lowering the levels of uric acid in the body fluids and tissues of mammals, preferably humans, and can thus be used for treatment of elevated uric acid levels associated with conditions including gout, tophi, renal insufficiency, organ transplantation and malignant disease. Specifically, pegylated uricase conjugates may be injected into a mammal having excessive uric acid levels by any of a number of routes, including intravenous, subcutaneous, intradermal, intramuscular and intraperitoneal routes. Alternatively, they may be aerosolized and inhaled. See Patton, J S, (1996) Adv Drug Delivery Rev 19:3-36 and U.S. Pat. No. 5,458,135.

The effective dose and duration of treatment with urate lowering therapy can depend on the level of uric acid suppression and the urate body burden. In a preferred embodiment, pegylated uricase, an effective urate lowering agent, is administered in a pharmaceutically acceptable excipient or diluent at 8 mg every two weeks. In another embodiment, pegylated uricase may be administered at 8 mg every four weeks. In yet another embodiment, pegylated uricase may be administered at 8 mg every three weeks.

Pharmaceutical formulations containing pegylated uricase can be prepared by conventional techniques, e.g., as described in Gennaro, A R (Ed.) (1990) Remington's Pharmaceutical Sciences, 18th Edition Easton, Pa.: Mack Publishing Co. Suitable excipients for the preparation of injectable solutions include, for example, phosphate buffered saline, lactated Ringer's solution, water, polyols and glycerol. Pharmaceutical compositions for parenteral injection comprise pharmaceutically acceptable sterile aqueous or non-aqueous liquids, dispersions, suspensions, or emulsions as well as sterile powders for reconstitution into sterile injectable solutions or dispersions just prior to use. These formulations may contain additional components, such as, for example, preservatives, solubilizers, stabilizers, wetting agents, emulsifiers, buffers, antioxidants and diluents.

Pegylated uricase may also be provided as controlled-release compositions for implantation into an individual to continually control elevated uric acid levels in body fluids. For example, polylactic acid, polyglycolic acid, regenerated collagen, poly-L-lysine, sodium alginate, gellan gum, chitosan, agarose, multilamellar liposomes and many other conventional depot formulations comprise bioerodible or biodegradable materials that can be formulated with biologically active compositions. These materials, when implanted or injected, gradually break down and release the active material to the surrounding tissue. For example, one method of encapsulating pegylated uricase comprises the method disclosed in U.S. Pat. No. 5,653,974, which is hereby incorporated by reference. The use of bioerodible, biodegradable and other depot formulations is expressly contemplated in the present invention.

The uricase used in pegylated uricase may comprise a mammalian uricase amino acid sequence truncated at the amino terminus or the carboxy terminus or both the amino and carboxy termini by about 1-13 amino acids and may further comprise an amino acid substitution at about position 46. The truncated uricase may further comprise an amino terminal amino acid, wherein the amino terminal amino acid is alanine, glycine, proline, serine, or threonine as described in co-pending PCT/US2006/013660 and U.S. provisional application Ser. No. 60/670,573, which are hereby incorporated herein by reference in their entireties.

The Phase 3 studies of pegylated uricase in Treatment Failure Gout and the First Application of Computer-Assisted Analysis of Digital Photographs for assessing tophus response was performed as described in Example 1.

Normalization of uric acid to <6.0 mg/dL was selected as the primary outcome measure to reflect the pharmacodynamic effect of pegylated uricase. It is known that persistently elevated plasma uric acid (PUA) or serum uric acid (SUA) levels result in deposition of uric acid in joints and soft tissues. As the total body burden of uric acid increases, signs and symptoms of gout result, including arthritis, characterized by recurrent painful gout flares, development of tophi and joint deformities with resultant chronic pain/inflammation and consequent loss of physical function.

Based on the pharmacodynamic effect of pegylated uricase on SUA, it was expected that clinical benefit in the TOPHACEOUS GOUT population would be observed by decreases in number of tophi, tender or swollen joints, fewer gout flares over time after an initial increase in flares associated with the initiation of urate-lowering therapy, and improvements in patient reported global assessments of disease activity, pain, physical function (measured by HAQ) and health-related quality of life (HRQOL, by SF-36). These were selected as important secondary outcome measures to demonstrate that sustained lowering of uric acid levels would be associated with clinically meaningful improvements.

### Example 1

### Material, Methods and Design of Clinical Study

### Phase III Efficacy Results

### Investigational Drug

Pegloticase, a pegylated uricase used in this example, consists of a recombinant mammalian uricase (primarily porcine, with C-terminal sequence from baboon uricase), conjugated with multiple strands of monomethoxy PEG of average molecular weight 10 kDa (10 K mPEG) per subunit of tetrameric enzyme (Kelly S J, et al. J Am Soc Nephrol 2001, 12:1001-1009; and Ganson N J, et al. Arthritis Res Ther 2005, 8(1):R12). It was manufactured by Savient Pharmaceuticals, Inc. (East Brunswick, N.J.) and supplied in vials containing 12.9 mg of pegloticase (233 Units, assayed as described below) in 1 mL of a phosphate buffer.

### Key Design Aspects.

The primary objective of these replicate studies was to demonstrate statistical significance in the number of patients receiving pegloticase compared with those receiving placebo in achieving serum uric acid concentrations <6 mg/dL for at least 80% of the time during months 3 and 6. Secondary outcomes included reductions in tophus burden and incidence/frequency of gout flares during months 4 to 6; tender or swollen joint counts; clinical global assessment of disease activity and patient reported outcomes: global assessment of disease activity, pain, physical function.

These were two, replicate randomized, multi-center, double-blind, 3-arm parallel treatment group, placebo-controlled trails of pegloticase, administered via intravenous infusion, in patients with hyperuricemia and symptomatic gout in whom conventional therapy was contraindicated or has been ineffective. Patients must have discontinued any uric acid-lowering agents for at least one week prior to receiving study drug, and refrain from using such agents throughout the study. Patients not already receiving prophylactic regimens of colchicine or non-steroidal antiinflammatory drug (NSAID) to prevent gout flares initiated such treatment at screening visit, unless medically contraindicated. After completing the study, patients had the option (and were encouraged) to continue active treatment for up to another 24 months by entering an Open Label Extension (OLE) protocol.

### Primary Efficacy Endpoint.

The primary efficacy endpoint was normalization of plasma uric acid (PUA) concentration to <6 mg/dL; using a predefined responder analysis, i.e., the proportion of patients with plasma uric acid (PUA) concentrations <6 mg/dL for ≧80% of the time during treatment Months 3 and 6.

### Secondary Efficacy Endpoint.

Based on a predefined pooled analysis of both identical phase 3 RCTs, the following secondary efficacy endpoints were assessed:

Reduction in tophus burden, using digital photography, in patients with evaluable tophi, i.e. “tophus evaluable population”, incidence and frequency of gout flares during months 4 to 6 of treatment, tender and/or swollen joint counts, clinician global assessment of disease activity and patient reported outcomes (PROs).

As the serum becomes supersaturated with urate, the material begins to deposit in the tissues. With some patients this not only leads to increased tissue stores of urate, but may lead to recurrent gout flares; however, in others this might lead to extensive tissue deposition which could lead to the development of a tophus (tophi) sitting in the periarticular tissues, on extensor surfaces, in the subperiorsteal space, in bursae, around the ear, or more rarely in the spinal cord, brain, or in organs.

The resolution of these tophi has not been demonstrated with available urate-lowering therapies alone or in combination with a uricosuric in randomized controlled trials.

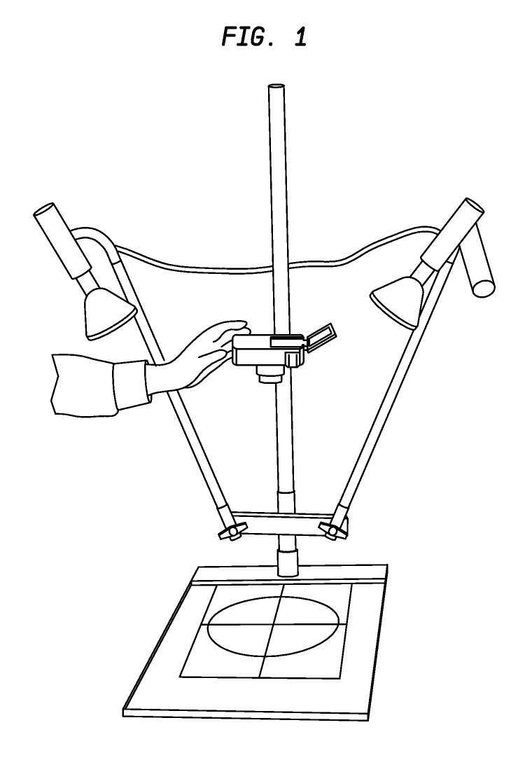

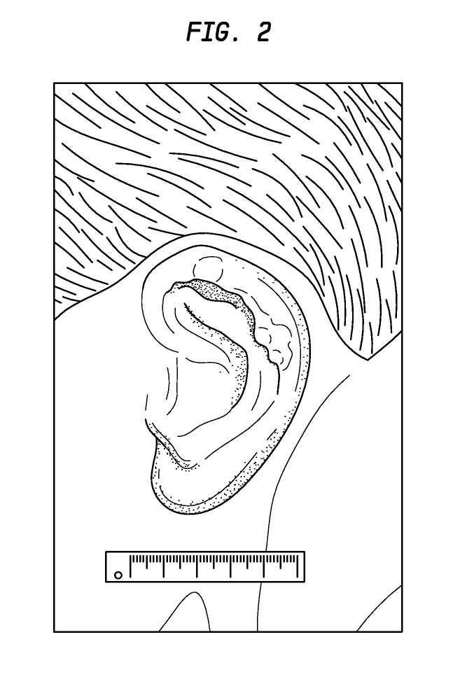

Baseline photographs of the hands and feet and other sites (up to 2 other representative sites) of baseline tophus lesion that could be photographed, e.g., elbow and knee, were obtained in each patient prior to initial study drug administration in a standardized manner were submitted to the digital imaging vendor. For these experiments, the following standardized equipment was used: Camera (calibrated and preset) and media card, light stand and lights, preprinted templates for placing hands and feet in standard positions, preprinted ruler allowing the linear dimension to be calibrated in the photographic rendering of the tophus using electronic calipers, labels and training manual and video. The proprietary software, MedStudio™, was used for electronic measurement and document management of the electronic images. For taking photographs, the light stand with camera and template for hands and feet is shown in FIG. 1. Further, FIG. 2 shows how calibration ruler is used for anatomic sites other than hands and feet. At each interval 2 or 3 photographs are taken to ensure an acceptable photo has been taken.

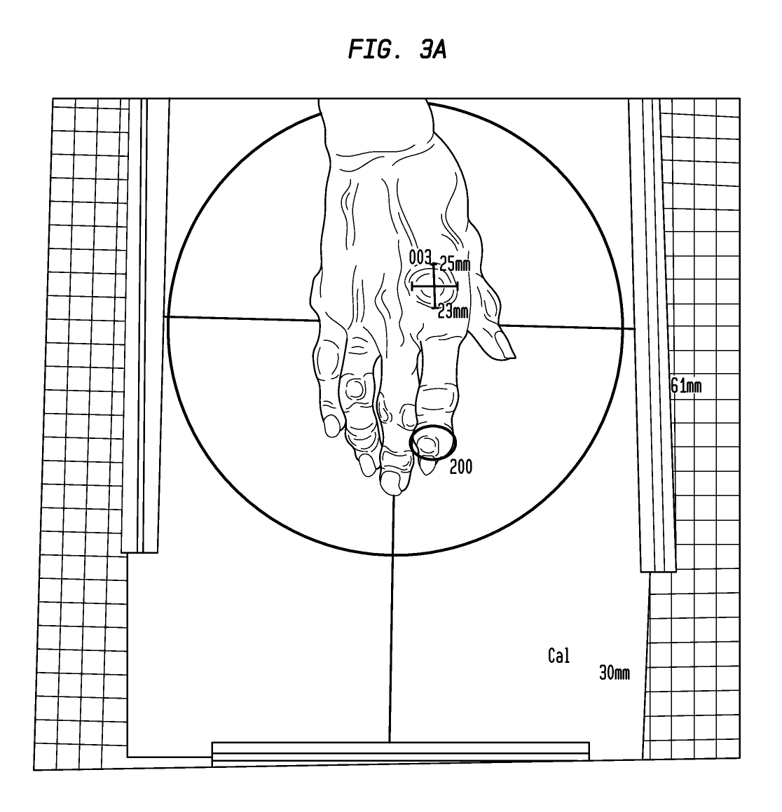

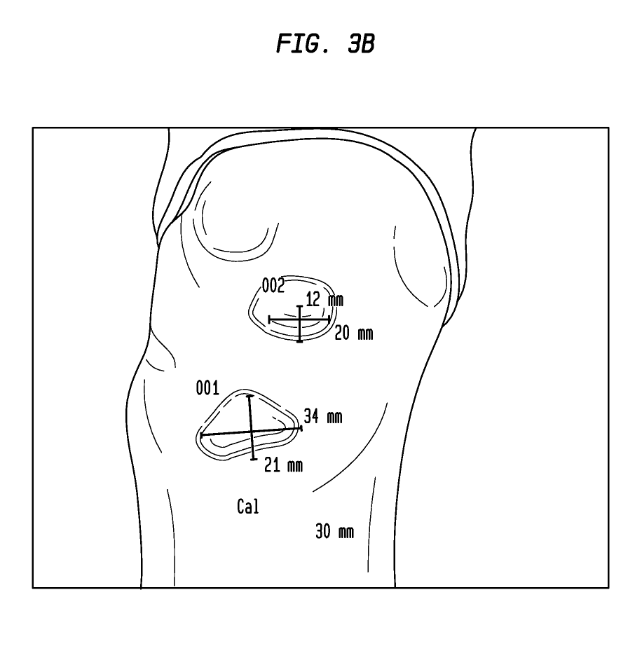

Further, CAPER (Computer-Assisted Photographic Evaluation in Rheumatology) was created to provide categorical scoring of tophus response recorded by photographic imaging. For these experiments, bi-dimensional measurements were considered to be more relevant for present application than the one-dimensional RECIST approach. FIGS. 3A and 3B shows the use of electronic calipers measuring the two longest diameters of measured tophi (perpendicular line) and the ellipse designates a tophus at the distal interphalangeal (DIP) joint with indistinguishable borders.

Tophi that were assessed were categorized as “measured” and “unmeasured” based on the Central Readers assessment of presence of distinguishable borders in the photographs. For these experiments, up to 5 measurable tophi, ≧5 mm at baseline in the longest dimension, with distinguishable borders in photographs were chosen by the central reader for assessment over the course of therapy. Further, up to 2 tophi representative of tophus burden which could not be precisely measured (e.g. due to location, shape or other factors) were also followed during the study—these semi-quantitatively assessed tophi must have been approximately ≧10 mm at baseline to allow reliable assessment of change in size.

For these experiments, central reader identified tophus margins, set the calibration of the calipers according the photographed ruler, and placed the electronic calipers at the edges of the longest diameter. Further, central reader identified margins of the longest perpendicular diameter and placed the electronic calipers at the edges. Finally, the computer measured the length of the two diameters set by the placement of the electronic calipers and calculated the area.

Individual categorical scores were determined for each measured target tophus as follows:

Complete Response (CR): 100% decrease in the area of the tophus from baseline;
Marked Response (MR): At least a 75% decrease in the area of the tophus from baseline;
Partial Response (PR): At least a 50% decrease in the area of the tophus from baseline;
Stable Disease (SD): Neither a 50% decrease nor a 25% increase in the area of the measurable tophus can be demonstrated;
Progressive Disease (PD): A 25% or more increase in the area of the tophus from Baseline and
Unable to Evaluate (UE): If a target tophus could not be measured or assessed at any of the post-baseline time intervals (e.g., due to a missing or poor quality photo), it was considered UE for that interval.

For the unmeasured tophus, the individual categorical scores were determined as follows:

Complete Response (CR): Disappearance of the tophus
Improved (I): An approximate 50% or greater reduction from baseline
Stable Disease (SD): Neither improvement nor progression from baseline can be determined
Progressive Disease (PD): ˜50% or more increase in the area of the tophus.

Follow-up photographs were obtained for tophi at weeks 13, 19 and 25, final visit or early termination. At each timepoint, these were assessed by the blinded central reader and compared to baseline, including new appearance of a tophus not evident at baseline. Photographs were read in “sequential locked read” format, programmatically controlled by software that prohibited the reader from changing the previous evaluation.

Most tophi were precisely assessed bidimensionally (using longest diameter and longest perpendicular to that diameter) and response of each individual tophus categorized according to change from baseline in area of each tophus at each visit. Some tophi were unable to be precisely quantified as their margins could not be precisely defined. These tophi were assessed for global change. Table 1 summarizes determinations of each individual tophus.

TABLE 1
Determinations of each individual tophus.

| Assessments | Measured Tophus* | Unmeasured Tophus** |
|---|---|---|
| Complete | 100% decrease in area | Dissapearance of tophus |
| Response (CR) | of tophus | |
| Marked | ≧75% decrease in area | |
| Response (MR) | of tophus | |
| Improved (I) | | ≧50% reduction in size |
| | | of tophus |
| Partial | ≧50% decrease in area | |
| Response (PR) | of tophus | |
| Stable | Neither 50% decrease nor | Neither improvement nor |
| Disease (SD) | 25% increase in area | progression from base- |
| | of tophus | line |
| Progressive | ≧25% increase in area | ≧50% increase in area |
| Disease (PD) | of tophus | of tophus |
| Unable to | tophus cannot be accurately | Tophus cannot be assessed |
| Evaluate (UE) | measured at any time point | for any reason at any time |
| | | point |

As shown in Table 2, overall response for a patient was based upon the best response among all tophi (measured and unmeasured) for that patient. Individual and overall tophus responses were summarized by visit; number of patients with resolution of any tophus and time to tophus resolution were summarized by treatment group.

TABLE 2
Definitions of tophus response in individual patients

| Overall | |
|---|---|
| Assessments | Determination of Overall Assessment |
| Complete | If CR (no PD) for either precisely measured or |
| Responder (CR) | globally measured tophus |
| Partial | If MR or PR (no CR, PD) for precisely measured or |
| Responder (PR) | improved for globally measured tophus |
| Stable | If SD (no CR, PR, PD) for either precisely |
| Disease (SD) | measured or globally measured tophus |
| Progressive | If PD for any precisely measured or globally |
| Disease (PD) | measured tophus, or if any new tophus appears |
| | during study treatment |
| Unable to | If UE for all precisely measured or globally |
| Evaluate (UE) | measured tophus |

Numbers of patients with an overall tophus response of CR (i.e., responder) were compared between each of the pegloticase dose groups against the placebo group using the Fisher's exact test. In addition, the overall tophus response such as CR, PR, SD, or PD were assigned an ordinal score of 1, 2, 3, or 4, and the two-sample Wilcoxon test were used to compare each of the pegloticase dose groups against the placebo group for the tophus assessment.

Time to tophus resolution was defined as the earliest time at which a complete resolution was demonstrated in one target tophus. Patients without tophus resolution were excluded from this analysis. Kaplan-Meier plots were presented by treatment for the time point stated above.

As shown in Table 3, treatment with pegloticase 8 mg q2 weeks demonstrated statistically significant reduction in tophus burden compared to placebo over time. At the first tophus response assessment visit at Week 13, 22% of patients experienced complete response of a target tophus (p=0.011); after 6 months (week 25) of pegloticase 8 mg 2q weeks treatment, 45% experienced CR (p=0.002).

TABLE 3
Assessment of overall tophus response in tophus

| | 8 mg Pegloticase | | |
|---|---|---|---|
| | Every 2 Weeks | Every 4 Weeks | Placebo |
| Overall Response of | (N = 62) | (N = 64) | (N = 29) |
| Tophus Assessment | n (%) | n (%) | n (%) |

Week 13 (Visit 11, Dose 7)

| | | | | | | |
|---|---|---|---|---|---|---|
| Complete Response (CR) | 10 | (21.7%) | 4 | (8.3%) | 0 | (0.0%) |
| Partial Response (PR) | 11 | (23.9%) | 9 | (18.8%) | 4 | (16.0%) |
| Stable Disease (SD) | 20 | (43.5%) | 28 | (58.3%) | 13 | (52.0%) |
| Progressive Disease (PD) | 5 | (10.9%) | 7 | (14.6%) | 8 | (32.0%) |

| | | | |
|---|---|---|---|
| Number of subjects with | 46 | 48 | 25 |
| evaluable tophi | | | |
| P-value¹ | 0.002 | 0.068 | — |
| P-value² | 0.011 | 0.292 | — |

Week 19 (Visit 14, Dose 10)

| | | | | | | |
|---|---|---|---|---|---|---|
| Complete Response (CR) | 16 | (36.4%) | 12 | (27.9%) | 2 | (7.7%) |
| Partial Response (PR) | 11 | (25.0%) | 9 | (20.9%) | 3 | (11.5%) |
| Stable Disease (SD) | 12 | (27.3%) | 19 | (44.2%) | 14 | (53.8%) |
| Progressive Disease (PD) | 5 | (11.4%) | 3 | (7.0%) | 7 | (26.9%) |

| | | | |
|---|---|---|---|
| Number of subjects with | 44 | 43 | 26 |
| evaluable tophi | | | |
| P-value¹ | 0.001 | 0.004 | — |
| P-value² | 0.010 | 0.063 | — |

Week 25 (Visit 20, 14 Days After Dose 12)

| | | | | | | |
|---|---|---|---|---|---|---|
| Complete Response (CR) | 18 | (45.0%) | 11 | (26.2%) | 2 | (8.0%) |
| Partial Response (PR) | 8 | (20.0%) | 10 | (23.8%) | 6 | (24.0%) |
| Stable Disease (SD) | 10 | (25.0%) | 16 | (38.1%) | 11 | (44.0%) |
| Progressive Disease (PD) | 4 | (10.0%) | 5 | (11.9) | 6 | (24.0%) |

| | | | |
|---|---|---|---|
| Number of subjects with | 40 | 42 | 25 |
| evaluable tophi | | | |
| P-value¹ | 0.002 | 0.061 | — |
| P-value² | 0.002 | 0.109 | — |

Final Visit

| | | | | | | |
|---|---|---|---|---|---|---|
| Complete Response (CR) | 21 | (40.4%) | 11 | (21.2%) | 2 | (7.4%) |
| Partial Response (PR) | 12 | (23.1%) | 12 | (23.1%) | 6 | (22.2%) |
| Stable Disease (SD) | 14 | (26.9%) | 21 | (40.4%) | 13 | (48.1%) |
| Progressive Disease (PD) | 5 | (9.6%) | 8 | (15.4%) | 6 | (22.2%) |

| | | | |
|---|---|---|---|
| Number of subjects with | 52 | 52 | 27 |
| evaluable tophi | | | |
| P-value¹ | 0.001 | 0.142 | — |
| P-value² | 0.002 | 0.200 | — |

¹An ordinal score (for categorical analysis) was assigned for each response and used to compute the P-value, which was based on two sample Wilcoxon test to compare corresponding pegloticase groups vs. placebo.
²P-value based on Fisher's exact test to compare percent of CR between corresponding pegloticase groups vs. placebo.

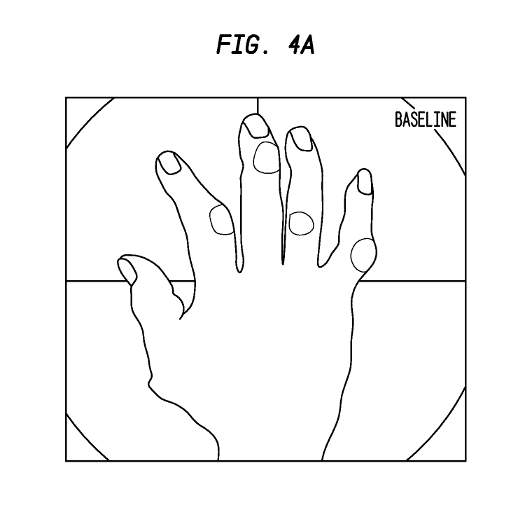

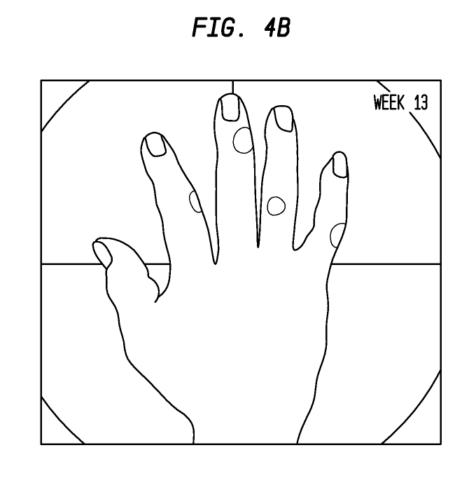

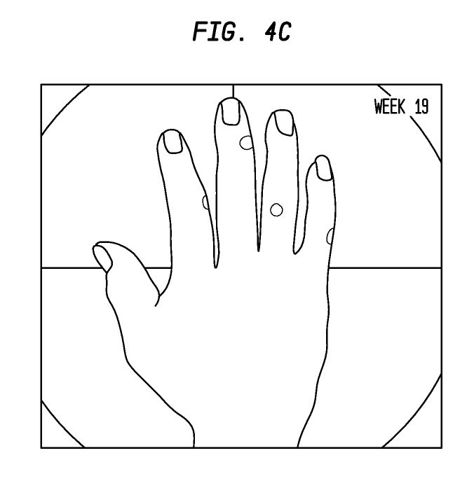

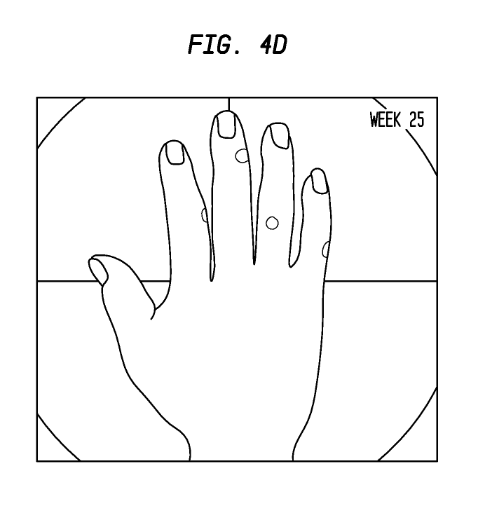

Further, FIGS. 4A-4D exemplifies a patient who received pegloticase for 2 weeks (q2) with a complete tophus resolution. Specifically, FIG. 4A depicts photographs of the patient's hand at baseline; FIG. 4B depicts photographs of the same hand at Week 13; FIG. 4C depicts photographs of the same hand at Week 19, and FIG. 4D depicts photographs of the same hand at Week 25. Note that the two target tophi at the metacarpophalangeal (MCP) joints have completely resolved by Week 19, as have all observed non-target tophi. Further, Week 25 is a close-up to optimally demonstrate complete resolution. In the 2 double-blind RCTs, there were 2 of 43 (5%) patients in the placebo group that had an overall CR at the final visit. This demonstrates the low rate of false positive responses using the CAPER method to measure tophus response to pegloticase.

With continued pegloticase in the OLE, further resolution of tophi was demonstrated. As shown in Table 4, interim data from the OLE study demonstrated that upon continued treatment with pegloticase, an additional 12 patients that received pegloticase demonstrated a CR in Overall Tophus Response for the first time in the OLE Study.

TABLE 4
Shift to Subjects's Overall Tophus Response from the Final Visit in the Double-Blind Study to Last Visit in OLE.

| | Double-Blind Period | | | | | | | | | | | |
|---|---|---|---|---|---|---|---|---|---|---|---|---|
| | 8 mg Pegloticase Every 2 Weeks | | | | 8 mg Pegloticase Every 4 Weeks | | | | Placebo | | | |
| | (N = 45) | | | | (N = 40) | | | | (N = 26) | | | |
| OLE | n (%) | | | | n (%) | | | | n (%) | | | |
| Period | CR | PR | SD | PD | CR | PR | SD | PD | CR | PR | SD | PD |

8 mg Pegloticase Every 2 Weeks (N = 68)

| | | | | | | | | | | | | |
|---|---|---|---|---|---|---|---|---|---|---|---|---|
| CR | 10 | 1 | 1 | 0 | 3 | 2 | 2 | 0 | 2 | 2 | 1 | 3 |
| | (41.7) | (4.2) | (4.2) | (0.0) | (18.8) | (12.5) | (12.5) | (0.0) | (11.8) | (11.8) | (5.9) | (17.6) |
| PR | 1 | 1 | 6 | 0 | 1 | 2 | 1 | 0 | 0 | 1 | 2 | 1 |
| | (4.2) | (4.2) | (25.0) | (0.0) | (6.3) | (12.5) | (6.3) | (0.0) | (0.0) | (5.9) | (11.8) | (5.9) |
| SD | 0 | 0 | 2 | 0 | 0 | 0 | 3 | 0 | 0 | 0 | 2 | 0 |
| | (0.0) | (0.0) | (8.3) | (0.0) | (0.0) | (0.0) | (18.8) | (0.0) | (0.0) | (0.0) | (11.8) | (0.0) |
| PD | 0 | 2 | 0 | 0 | 1 | 0 | 0 | 1 | 0 | 0 | 1 | 2 |
| | (0.0) | (8.3) | (0.0) | (0.0) | (6.3) | (0.0) | (0.0) | (6.3) | (0.0) | (0.0) | (5.9) | (11.8) |

8 mg Pegloticase Every 4 Weeks (N = 43)

| | | | | | | | | | | | | |
|---|---|---|---|---|---|---|---|---|---|---|---|---|
| CR | 6 | 3 | 0 | 1 | 5 | 1 | 1 | 0 | 0 | 0 | 2 | 0 |
| | (46.2) | (23.1) | (0.0) | (7.7) | (33.3) | (6.7) | (6.7) | (0.0) | (0.0) | (0.0) | (33.3) | (0.0) |
| PR | 0 | 1 | 0 | 0 | 0 | 2 | 0 | 0 | 0 | 2 | 0 | 0 |
| | (0.0) | (7.7) | (0.0) | (0.0) | (0.0) | (13.3) | (0.0) | (0.0) | (0.0) | (33.3) | (0.0) | (0.0) |
| SD | 0 | 0 | 1 | 0 | 0 | 0 | 2 | 0 | 0 | 0 | 2 | 0 |
| | (0.0) | (0.0) | (7.7) | (0.0) | (0.0) | (0.0) | (13.3) | (0.0) | (0.0) | (0.0) | (33.3) | (0.0) |
| PD | 0 | 0 | 0 | 1 | 0 | 1 | 2 | 1 | 0 | 0 | 0 | 0 |
| | (0.0) | (0.0) | (0.0) | (7.7) | (0.0) | (6.7) | (13.3) | (6.7) | (0.0) | (0.0) | (0.0) | (0.0) |

Further, as shown in Table 4, among the previously placebo-treated patients who switched to pegloticase treatment in the OLE Study, there were eight patients that demonstrated an Overall Tophus Response of CR. Based on the results presented above, it had been concluded that treatment with pegloticase, every two weeks (q2) was associated with statistically significantly more complete responders of tophi in pooled analysis and both RCTs compared with placebo. Additionally, the number of patients with overall complete tophus response increased with increased duration of pegloticase.

What is claimed is:

1. A method for accessing and/or quantitating tophus response during a urate lowering therapy in a patient comprising:
a) obtaining a photograph of each baseline tophus before urate-lowering treatment and each tophus after said treatment;
b) measuring the longest dimension and the perpendicular dimension of said tophus before and after said treatment;
c) calculating the area of said tophus before and after said treatment by multiplying said longest dimension and said perpendicular dimension;
d) comparing the tophus area before and after said urate-lowering treatment; and
e) indicating that a change in tophus area from said baseline correlates with therapeutic efficacy.

2. The method according to claim 1 wherein the urate-lowering therapy administered to a patient in need thereof is pegylated uricase therapy.

3. The method according to claim 2 wherein pegylated uricase is administered at a dosage of 8 mg every 2 weeks.

4. The method according to claim 2, wherein pegylated uricase is administered at a dosage of 8 mg every 4 weeks.

5. The method according to claim 2, wherein the tophus photographs are taken after 25 weeks of pegylated uricase treatment.

6. The method according to claim 2, wherein the tophus photographs are taken after 19 weeks of pegylated uricase treatment.

7. The method according to claim 2, wherein the tophus photographs are taken after 13 weeks of pegylated uricase treatment.

8. The method according to claim 1 wherein said patient is suffering from gout.

9. The method according to claim 9 wherein said gout is refractory to conventional therapy.

10. The method according to claim 9 wherein said gout is chronic or tophaceous.

11. The method according to claim 2 wherein at least 75% decrease in the tophus area from baseline correlates with marked response to pegylated uricase therapy

12. The method according to claim 2 wherein 100% decrease in the tophus area from baseline correlates with complete response to pegylated uricase therapy.

13. The method according to claim 2 wherein at least 75% decrease in the tophus area from baseline correlates with marked response to pegylated uricase therapy

14. The method according to claim 2 wherein at least 50% decrease in the tophus area from baseline correlates with partial response to pegylated uricase therapy.

15. The method according to claim 2 wherein at least 25% increase in the tophus area from baseline correlates with progressive disease.

16. The method according to claim 1 wherein the tophus is characterized as measurable.

17. The method according to claim 1 wherein the tophus is characterized as unmeasured.
